(19) United States
(12) Patent Application Publication
(10) Pub. No.: US 2008/0118480 A1
(43) Pub. Date: May 22, 2008

(54) Methods and Compositions for Directing Migration of Neural Progenitor Cells

(75) Inventors: Huanxiang Zhang, Beijing (CN); Laszlo Vutskits, La Corix-de-Rozon (CH); Michael Pepper, Pretoria (ZA); Jozsef Zoltan Kiss, Plan-les-Ouates (CH)

Correspondence Address: LUCAS & MERCANTI, LLP, 475 PARK AVENUE SOUTH, 15TH FLOOR, NEW YORK, NY, 10016, US

(73) Assignee: IMCLONE SYSTEMS INCORPORATED, New York, NY (US)

(21) Appl. No.: 10/583,786

(22) PCT Filed: Dec. 20, 2004
(86) PCT No.: PCT/US04/43482

§ 371 Date: Sep. 14, 2007

Related U.S. Application Data
(60) Provisional application No. 60/531,118, filed on Dec. 19, 2003.

(51) Int. Cl. A61K 48/00; A61P 25/00; A61K 38/16; C12N 5/00

(52) U.S. Cl. 424/93.21; 435/375; 514/12

(57) ABSTRACT

Method for modulating the migration of neural progenitor cells in a mammal by exposing the cells to a VEGFR-2 ligand and FGF-2. Methods of treating neurological disorders by exposing the mammal to a VEGFR-2 ligand in the presence of FGF-2 are also provided. A composition including a biocompatible matrix associated with FGF-2 is also provided.

### FIELD OF THE INVENTION

The present invention provides methods and compositions for modulating migration of neural progenitor cells and methods for treating conditions involving loss or injury of neural cells and for treating neuronal migration disorders.

### BACKGROUND OF THE INVENTION

Migration of immature neurons during development is essential for the proper formation of the nervous system. In the mammalian brain, most neurons are generated within proliferative zones around the ventricle from where immature precursors migrate to specific sites in the cerebral wall. A variety of clinical syndromes, including various forms of Lissencephalies, are related to deficient migration of neural cells. The consequences of these malformations include mental retardation, epilepsy, paralysis and blindness. Genetic studies of some of these perturbations have provided some understanding of the regulation of neuronal migration, which has rapidly expanded over the past ten years.

In addition to playing a key role in early development, neuronal migration is also important for the adult brain. For example, in the brain of songbirds, neurogenesis and neuronal migration are required for structural plasticity and learning throughout adulthood. Recent evidence suggests that undifferentiated multipotential progenitors also exist in the adult mammalian brain and during adult neurogenesis, as well as during the continuous neuronal replacement that occurs at specific sites in the rostral subventricular zone-olfactory bulb system and the dentate gyrus.

Finally, cell migration plays a central role in wound repair. Although the intrinsic capacity of the adult mammalian brain to replace lost or damaged neurons is very limited, migration of neural progenitor cells and cell replacement has been reported after administration of various factors.

Considerable effort has recently been focused on understanding the factors and mechanisms involved in the navigation of immature neurons to their final destination. Highly conserved families of attractive and repulsive molecules are coordinately regulated in order to guide neurons to their final destination. These molecules include netrins, semaphorins, ephrins, Slits and various neurotrophic factors. Compared to migration of post-mitotic immature neurons, little is known about the factors and mechanisms that direct the migration of neural stem cells and undifferentiated neural progenitor cells. In one study, placental derived growth factor (PDGF) was shown to attract FGF-2-stimulated neural progenitor cells in a transfilter migration assay.

Identifying candidate molecules that play a role in neural progenitor cell migration is crucial not only for understanding proper tissue formation during development, but also for developing methods for directing undifferentiated neural progenitor cells to achieve structural brain repair.

### SUMMARY OF THE INVENTION

In an embodiment, the present invention provides a method for modulating the migration of neural progenitor cells comprising exposing the cells to FGF-2 and a VEGFR-2 ligand. In another embodiment, the present invention provides a method for treating a mammal having a disorder involving loss or injury of neural cells comprising exposing the mammal to a VEGFR-2 ligand in the presence of FGF-2 to stimulate migration of neural progenitor cells to the site of neural loss or injury.

In another embodiment, the present invention provides a method for treating a mammal having a neural tissue site with a deficient neuronal population. The method comprises exposing the mammal to a VEGFR-2 ligand in the presence of FGF-2 to stimulate migration of neural progenitor cells to the neural tissue site.

In another embodiment, the present invention provides a method for modulating the migration of neural progenitor cells comprising exposing the cells to a compound capable of increasing or maintaining the expression of VEGFR-2 on the cells and exposing the cells to a VEGFR-2 ligand.

In another embodiment, the present invention provides pharmaceutical compositions comprising a VEGFR-2 ligand, FGF-2, and a carrier.

In another embodiment, the present invention provides a composition comprising a biocompatible matrix comprising FGF-2. Preferably, the biocompatible matrix also includes a VEGFR-2 ligand.

### BRIEF DESCRIPTION OF THE DRAWINGS

FIGS. 1A-F show morphological and immunocytochemical characterization of neural progenitor cells cultured in the presence of FGF-2. FIG. 1A shows contrast images of the cells at day 4 and FIG. 1B at day 6. FIG. 1C shows that after the sixth day in culture, the majority of cells are immunopositive for nestin, indicating that they are undifferentiated neural progenitor cells. FIG. 1D shows that BrdU incorporation indicates that the majority of cells are proliferating. The rare cells that are positive for the neuronal marker (TuJ, arrow) are nonproliferative. FIGS. 1E and 1F show that five days after the withdrawal of FGF-2, cells have differentiated into GFAP containing astrocytes (FIG. 1E), Tuj positive neurons (FIG. 1E) and GalC positive oligodendrocytes (FIG. 1F). Cell nuclei were counterstained with Hoechst 33342 in FIGS. 1C, 1E and 1F. Scale bars, 80 μm in FIGS. 1A and 1B, 30 μm in FIG. 1C; 19 μm in FIG. 1D; 30 μm in FIGS. 1E and 1F.

FIGS. 2A-F demonstrate chemotaxis of neural progenitor cells stimulated by VEGF. FIG. 2A is a schematic representation of a Dunn chamber (top view) with the overlying coverslip, showing the position of the inner well, bridge and outer well. In FIG. 2B, cells over the annular bridge between the inner and outer wells of the chamber can be observed under phase-contrast optics. Cell migration was recorded continuously by time-lapse frame grabbing and the migration tracks were plotted in scatter diagrams shown in FIGS. 2C, 2D, 2E, and 2F. The starting point for each cell is at the intersection between the X and Y axes (0,0), and data points indicate the final positions of individual cells at the end of the 2-hour recording period. Chemotaxis was tested by placing VEGF (FIG. 2C) or FGF-2 (FIG. 2E) in the outer well. The direction of the gradient is vertically upwards. As shown in FIG. 2C and FIG. 2E, neural progenitor cells undergo chemotaxis and display a clear directionality of migration in the presence of VEGF (FIG. 2C), but not an FGF-2 (FIG. 2E) gradient. For chemokinesis (FIGS. 2D and 2F), equal amounts of VEGF or FGF-2 were added in both inner and outer wells of the chamber. Arrow in FIG. 2B indicates the direction of the outer well of the Dunn chamber. Scale bar, 50 μm. FIGS. 3A-D show migration tracks of neural progenitor cells.

FIG. 3A provides phase contrast photos showing a representative cell (*) migrating up a VEGF gradient. Arrow indicates the source of VEGF. FIG. 3B shows migration tracks of 4 representative cells in the presence of a VEGF concentration gradient. The starting point for each cell is at the intersection between the X and Y axes (0, 0) and the source of VEGF is at the top. FIG. 3C are phase contrast photos showing a neural progenitor cell that randomly migrates in a uniform concentration of VEGF. FIG. 3D shows migration tracks of 4 representative cells that migrate randomly under conditions of uniform VEGF distribution. The starting point for each cell is at the intersection between the X and Y axes (0, 0).

FIGS. 4A-B show the migration speed (μm/hour) (FIG. 4A) and forward migration index (FMI) values (FIG. 4B) under different conditions. Cell migration speed was calculated for each time-lapse interval and the mean speed was derived for a period of 2 hours. Data are shown as mean±SEM from at least 3 independent experiments. FMI values can be either positive or negative, depending on the direction in which the cells migrate. P is less than 0.01 by two-tailed unpaired t-test, which is significantly different from chemokinesis or an FGF-2 gradient.

FIGS. 5A-B show VEGF receptor expression in neural progenitor cells. In FIG. 5A, total cellular RNA was isolated and VEGF receptor MRNA expression was assessed by RNase protection analysis. Purified ³²P-labeled rat cRNA probes (probe) were hybridized to hybridization mix (probe+h.m.), yeast tRNA, or total RNA from cells grown in FGF-2 or starved of FGF-2 for 12 hours. Rat acidic ribosomal protein P0 was used as an internal control and the positive control was rat lung. In FIG. 5B, quantitative analysis of VEGFR-1 and VEGFR-2 expression is shown in cells cultured in the presence of FGF-2 or starved of FGF-2 for 12 hours. P is less than 0.01 by two-tailed unpaired t-test, which is significantly different from cells in FGF-2 (n=3 experiments).

FIGS. 6A-D show VEGF stimulated chemotaxis of neural progenitor cells through VEGFR-2. FIG. 6A shows the migation patterns of neural progenitor cells under control conditions or in the presence of VEGF receptor blockers. Cells treated with the VEGFR-2 blocking antibody (DC101) lost the chemotactic response to VEGF. In contrast, the VEGFR-1 blocking antibody (MF1) did not affect progenitor migration. FIG. 6B shows speed and FMI under different migration conditions. FIGS. 6C and 6D show migration tracks of representative cells (4 each condition) exposed to a VEGF concentration gradient, in the presence of either VEGFR-2 blocking antibody (FIG. 6C) or control (polysialic acid blocking) antibody (FIG. 6D). The starting point for each cell is at the intersection between the X and Y axes (0, 0) and the source of VEGF is at the top in the gradient condition. P is less than 0.01 by two-tailed unpaired t-test, which is significantly different from DC101-treated cells.

FIGS. 7A-E show FGF-2 enhanced ability of neural progenitor cells to chemotactically respond to a VEGF gradient. In FIG. 7A, for a first group, FGF-2 was withdrawn at day 5 for 12 hours, then cells were exposed to a VEGF gradient. In FIG. 7B, a second group was further cultured in the presence of FGF-2 after the 12-hour starvation period for 8 hours and then tested in a VEGF gradient In FIG. 7C, the final positions of the cells after 2 hours of migration is indicated, with the starting point for each cell at (0, 0) and the source of VEGF at the top. FIG. 7D shows speed and FMI. Data are shown as mean±SEM from 4 independent experiments. After 12 hours of FGF-2 starvation, cells lose their chemotactic response to the VEGF gradient. The starved neural progenitor cells resume their chemotactic response to VEGF upon re-addition of FGF-2 to the cultures for 8 hours (FIG. 7C). FIG. 7E shows VEGFR-2 expression in neural progenitor cells cultured in FGF-2 or starved of FGF-2 for 12 hours. Western blot analysis was performed on immunoprecipitates with an anti-VEGFR-2 antibody. P is less than 0.01 by two-tailed unpaired t-test.

FIGS. 8A-F show the effect of VEGF on neural progenitor cells migrating from subventricular zone (SVZ) explants. SVZ explants were co-cultured with VEGF-secreting C₂C₁₂ cells and/or mock-transfected C₂C₁₂ cells in collagen gel matrices in the presence (FIGS. 8A, 8B, 8D, 8E, and 8F) or absence (FIG. 8C) of FGF-2. In FIG. 8A, in the presence of FGF-2, neural progenitor cells migrate out of the SVZ explant in an asymmetric manner, with many more cells on the side of the VEGF-secreting C₂C₁₂ cells than on the side of control C₂C₁₂ cells. In FIG. 8B, neural progenitor cells migrate out of the SVZ explant symmetrically when cultured with control C₂C₁₂ cells on both sides. In FIG. 8C, in the absence of FGF-2, few to no cells migrate out of the SVZ explant. FIG. 8D is a high power photograph that shows the SVZ explant on the side of control C₂C₁₂ cells. FIG. 8E is a high power photograph that shows many neural progenitor cells migrating out of the SVZ explant toward VEGF-secreting C₂C₁₂ cells. In FIG. 8F, cells migrating out of the SVZ explant are positive for nestin, a marker for undifferentiated neural progenitor cells. Scale bar, 700 μm in FIGS. 8A, 8B and 8C; 100 μm in FIGS. 8D and 8E; 50 μm in FIG. 8F.

### DETAILED DESCRIPTION OF THE INVENTION

According to the present invention, vascular endothelial growth factor-2 (VEGFR-2) ligands, such as VEGF, VEGF-E, and VEGF-C/D_{ΔNΔC}, are chemoattractants for neural progenitor cells that express VEGFR-2, wherein migration of neuronal progenitor cells in response to a VEGFR-2 ligand is dependent on exposure of the cells to fibroblast growth factor-2 (FGF-2). The present invention provides a method for modulating the migration of neural progenitor cells by exposing the cells to FGF-2 and a VEGFR-2 ligand. Although not wishing to be bound by theory, it is believed that the FGF-2 maintains and/or increases expression of VEGFR-2 on the neural progenitor cells, to which either an endogenous or exogenous VEGFR-2 ligand binds. The cells can be exposed to an exogenous or endogenous VEGFR-2 ligand. For example, the cells can be exposed to an exogenous VEGFR-2 ligand when endogenous VEGF-2 ligands are not up-regulated or are otherwise present in an insufficient amount in the mammal to stimulate migration of the neural progenitor cells. The cells can be exposed to the VEGFR-2 ligand either before, after, or concurrently with exposure to the FGF-2.

In addition to expressing VEGFR-2, neural progenitor cells of the present invention express nestin and do not display antigenic markers for neuron- or glia-restricted precursor cells, such as, for example, PSA-NCAM, doublecortin, NEuN, NG2, or A2B5 and endothelial cell markers, such as, for example, von Willebrand factor and RECA-1. The neural progenitor cells may also express VEGFR-1 and preferably do not express VEGFR-3.

The present invention also provides a method of modulating migration of neural progenitor cells comprising exposing the cells to a compound capable of increasing or maintaining the expression of VEGFR-2 on the neural progenitor cells and exposing the cells to a VEGF-2 ligand. Non-limiting examples of compounds that are capable of increasing or maintaining the expression of VEGFR-2 includes FGF-2. Other compounds can be determined by screening for compounds capable of increasing or maintaining VEGFR-2 expression. Such screens may be performed by exposing neural progenitor cells to test compound, followed by assaying for the level of VEGFR-2 expression. Such expression may be detected using VEGFR-2 antibodies or labeled ligand.

The present invention also provides for compositions comprising an effective amount of FGF-2 and VEGFR-2, and a pharmaceutically acceptable carrier. In this embodiment, pharmaceutically acceptable means approved by a regulatory agency of the Federal or a state government or listed in the U.S. Pharmacopeia or other generally recognized pharmacopeia for use in animals, and more particularly in humans. The term carrier refers to a diluent, adjuvant, excipient, or vehicle with with the FGF-2 and VEGFR-2 is administered. Examples of suitable carriers are described in “Remington's Pharmaceutical Sciences” by E. W. Martin.

The present invention also provides a composition comprising a biocompatible matrix comprising FGF-2 and preferably also a VEGFR-2 ligand. The biocompatible matrix can be fabricated from natural or synthetic materials so long as the material does not produce an adverse or allergic reaction when administered to the mammal and can be administered into the nervous system. The matrix may be fabricated from non-biodegradable or biodegradable polymers. Non-limiting examples of non-biodegradable polymers include ethylene vinyl acetate, poly(meth)acrylic acid, polyamides, copolymers and mixtures thereof. Non-limiting examples of biodegradable materials include polyesters such as polyglycolides, polylactides, and polylactic polyglycolic acid copolymers (“PLGA”); polyethers such as polycaprolactone (“PCL”); polyanhydrides; polyakyl cyanocrylates such as n-butyl cyanoacrylate and isopropyl cyanoacrylate; polyacrylamides; poly(orthoesters); polyphosphazenes; polypeptides; polyurethanes; and mixtures of such polymers. The matrix may take the form of a sponge, implant, tube, lyophilized component, gel, patch, powder or nanoparticles or any other form that can be administered into the nervous system. When a VEGFR-2 ligand is added to the matrix, preferably the matrix allows for formation of a concentration gradient of the VEGFR-2 ligand. The matrix may further include one or more other suitable chemotactic or neurotrophic factors, such as growth factors (e.g., PDGF, NFG), netrins, semaphorins, ephrins, and Slits, for example. The composition comprising the biocompatible matrix can also include neural progenitor cells for transplantation of exogenous neural progenitor cells to the mammal receiving the composition. The neural progenitor cells may be derived from the mammal to be treated or from another source.

The present invention also provides a method of treating mammals having certain neurological disorders or conditions. For example, in one embodiment, the present invention provides a method of treating a mammal having a condition involving loss or injury of neural cells (including both neurons and glial cells). The method comprises exposing the mammal to a VEGFR-2 ligand and FGF-2 to stimulate migration of neural progenitor cells to the site of neural cell loss or injury. Non-limiting examples of conditions involving loss or injury of neural cells are brain injury caused by stroke, ischemia, anoxia or head trauma, for example.

In another embodiment, the present invention provides a method of treating disorders in a mammal having a neural tissue site with a deficient neuronal population by exposing the mammal to a VEGFR-2 ligand and FGF-2 to stimulate migration of neural progenitor cells to the deficient neural tissue site. Such disorders, characterized by certain neural tissue having a deficient neuronal population include those resulting in birth defects caused by the abnormal migration of neurons in the developing nervous system. Such abnormal migration of neurons results in incorrect positioning of neurons resulting in certain neural tissue sites lacking the necessary population of neurons. These disorders result in structurally abnormal or missing areas of the brain, for example, in the cerebral hemispheres, cerebellum, brainstem, or hippocampus, for example. Structural abnormalities as a result of such abnormal migration include, for example, schizocephaly, porencephaly, lissencephaly, agyria, macrogyria, pachygyria, microgyria, micropolygyria, neuronal heterotopias, ageneis of the corpus callosum, and agenesis of the cranial nerves. The present invention provides methods for treating such disorders by directing neural progenitor cells to the proper sites of the developing nervous system. For example, if neurons are not migrating to the cerebellum resulting in the cerebellum having a deficient population of neurons, the method of the present invention provides a means for stimulating the migration of neural progenitor cells to the cerebellum.

Methods of treating neurological disorders or conditions according to the present invention, may be used to stimulate endogenous neural progenitor cells and/or alternatively to stimulate exogenous neural progenitor cells transplanted into the mammal. Exposing the mammal to a VEGFR-2 ligand and FGF-2, according to these methods of the present invention, includes exposing the neural progenitor cells to an endogenous or exogenous VEGFR-2 ligand and endogenous or exogenous FGF-2. For example, an exogenous VEGFR-2 can be actively administering to the mammal if endogenous VEGF-2 ligands are not up-regulated or are otherwise present in an insufficient amount in the mammal to stimulate migration of the neural progenitor cells. The VEGFR-2 ligand can be administered before, after, or concurrently with exposure to FGF-2. In the lesion context, administration of a VEGFR-2 ligand may be unnecessary since endogenous VEGF may be up-regulated in the mammal. Likewise, exogenous FGF-2 can be actively administered to the mammal if endogenous FGF-2 is not present in sufficient amounts to stimulate migration of the neural progenitor cells.

The mammal can be exposed to the FGF-2, VEGFR-2 ligand and/or neural progenitor cells by any method known in the art. For example, the mammal can be exposed to these substances by direct administration via a catheter to the neural site in need of the neural progenitor cells or, in the case of stimulating migration of endogenous neural progenitor cells, to the neural site where the endogenous neural progenitor cells are located. In a preferred embodiment, the FGF-2, VEGFR-2 ligand, and/or endogenous neural progenitor cells are administered as part of composition comprising a biocompatible matrix, as described above. Further, the methods may further comprise administering to the mammal one or more other suitable chemotactic or neurotrophic factors, such as, for example, growth factors (e.g., PDGF, NFG), netrins, semaphorins, ephrins, and Slits.

The identification of neurological disorders treatable by the methods of the present invention is well within the ability and knowledge of one skilled in the art. For example, a clinician skilled in the art can readily determine, for example, by the use of clinical tests, diagnostic procedures, and physical examination, if an individual suffers from neuronal injury or loss or a neuronal migration disorder and is therefore a candidate for exposure to a VEGFR-2 ligand and FGF-2, according to the present invention.

The mammal can be exposed to the VEGFR-2 ligand and the FGF-2 in amounts sufficient to direct migration of neural progenitor cells. Data obtained from cell culture assays and animal studies can be used in formulating a range of dosage for use in mammals, including, for example, humans. The amounts that will be effective in the treatment of a particular disorder or condition will depend on the nature of the disorder or condition and can be determined by standard clinical techniques. In addition, in vitro assays may optionally be employed to help identify optimal dosage ranges. Amounts effective for this use will depend, for example, upon the severity of the disorder. Dosing schedules will also vary with the disease state and status of the patient, and will typically range from a single bolus administration or continuous infusion to multiple administrations per day, or as indicated by the treating physician and the patient's condition. It should be noted, however, that the present invention is not limited to any particular dose.

The present invention also provides a pharmaceutical pack or kit comprising one or more containers filled with FGF-2 and/or VEGFR-2.

In embodiments where neural progenitor cells are transplanted into the mammal, a population of neural progenitor cells can be isolated from a mammalian donor by methods known in the art. For example, neural progenitor cells can be isolated in vitro by dissecting out a region of fetal or adult neural tissue that has been demonstrated to contain dividing cells in vivo such as, for example, the subventricular zone (SVZ) or the hippocampus in adult brains and a larger variety of structures in the developing brain such as, for example, the hippocampus, cerebral cortex, cerebellum, neural crest, and basal forebrain. The neural tissue can then be disaggregated and the dissociated cells exposed to a high concentration of mitogens such as FGF-2 or epidermal growth factor-2 (EGF) in a defined or supplemented medium on a matrix as a substrate for binding. (Such methods further described in M. Alison et al. J. Hepatol. 26, 343 (1997) and J. M. W. Slack, Development, 121, 1569 (1995), both of which are incorporated by reference herein). The dissociated cells can then be exposed to molecules that bind specifically to antigen markers characteristic of the neural progenitor cells of the present invention such as nestin, or VEGFR-2. The cells that express these antigen markers bind to the binding molecule allowing for isolation of neural progenitor cells. If the neural progenitor cells do not internalize the molecule, the molecule may be separated from the cell by methods known in the art. For example, antibodies may be separated from cells by short exposure to a solution having a low pH or with a protease such as chymotrypsin.

The molecule used for isolating the population of neural progenitor cells may be conjugated with labels that expedite the identification and separation of the neural progenitor cells. Examples of such labels include magnetic beads and biotin, which may be identified or separated by means of its affinity to avidin or streptavidin and fluorochromes.

Methods for removing unwanted cells by negative selection can also be used. For example, the cells can be exposed to molecules that bind specifically to antigen markers that are not characteristic of the neural progenitor cells of the present invention such as PSA-NCAM, doublecortin, NeuN, NG2, A2B5 and cells that bind to these molecules can be removed.

Once the neural progenitor cells are isolated, they can be transplanted and grafted into the desired site of the nervous system of the mammal by methods known in the art, such as the methods described in Flax et al., “Engraftable human neural stem cells respond to developmental cues, replace neurons, and express foreign genes” Nature Biotech., 16:1033-1039 (1998); Uchida and Buck, “Direct isolation of human central nervous system stem cells,” Proc Natl Acad Sci USA, 97: 14720-14725 (2000); Brustle et el., “Chimeric brains generated by intraventricular transplantation of fetal human brain cells into embryonic rats,” Nature Biotech, 16: 1040-1044 (1998); and Fricker et al., “Site-specific migration and neuronal differentiation of human neural progenitor cells after transplantation in the adult brain,” J. Neurosci, 19: 5990-6005 (1999), all of which are incorporated by reference herein.

### EXAMPLES

### Example 1

### Isolation and Culture of Neural Progenitor Cells

The SVZ was dissected from coronal slices of newborn rat brains, dissociated mechanically and trypsinized according to methods known in the art (See Lim et al. “Noggin antagonizes BMP signaling to create a niche for adult neurogenesis, Neuron, 28: 713-726 (2000), which is incorporated by reference herein). SVZ progenitors were purified using percoll gradient centrifugation according to methods known in the art (See Lim et al., 2000) and seeded onto matrigel (0.24 mg/cm²)- or laminin-coated coverslips. Isolated cells were allowed to grow in Neurobasal medium supplemented with 20 ng/ml FGF-2, 1×B27, 2 mM glutamate, 1 mM sodium pyruvate, 2 mM N-acetyl-cysteine, and 1% penicillin-streptomycin. Cultures were fed every three days with fresh medium containing 20 ng/ml FGF-2.

Immunostaining of cultures was performed according to procedures known in the art (See Wang et al. “Functional N-methyl-D-aspartate receptors in O-2A glial precursor cells: a critical role in regulating polysialic acid-neural cell adhesion molecule expression and cell migration,” J. Cell Biol., 135:1565-1581 (1996); Vutskits et al. “PSA-NCAM modulates BDNF-dependent survival and differentiation of cortical neurons, Eur. J. Neurosci, 13: 1391-1402 (2001), both of which are incorporated by reference herein). The following primary antibodies and dilutions were used: mouse monoclonal antibody against nestin (Biogenesis, UK, 1:300 dilution); mouse monoclonal antibody against A2B5 (described in Eisenbarth et al. “Monoclonal antibody to a plasma membrane antigen of neurons,” Proc. Natl. Acad. Sci. USA, 76:4913-4917 (1979), which is incorporated by reference herein); hybridoma supernatant, ATCC, Rockville, Md., 1:5 dilution); Men B (Meningococcus group B) mouse IgM monoclonal antibody (1:500 dilution) that specifically recognizes a 2-8-linked PSA with chain length superior to 12 residues (described in Rougon et al., “A monoclonal antibody against Meningococcus group B polysaccharides distinguishes embryonic from adult N-CAM, J. Cell Biol., 103: 2429-2437 (1986), which is incorporated by reference herein); anti-GalC (described in Ranscht et al. “Development of oligodendrocytes and Schwaqnn cells studies with a monoclonal antibody against galactocerebroside,” Proc. Natl. Acad. Sci. USA, 79:2709-2713 (1982), which is incorporated by reference herein), mouse IgM monoclonal antibody (culture supernatant, 1:5 dilution); Tuj mouse monoclonal antibody directed against β-tubulin isotype III (1:400 dilution) (Sigma, Saint Louis, Mo.); a rabbit polyclonal antibody to GFAP (Dakopatts, Copenhagen, Denmark, 1:200 dilution); a rabbit polyclonal antibody against NG2 (Chemicon International, California, 1:400 dilution); a goat polyclonal antibody against Doublecortin (Santa Cruz Biotecnology, 1:300 dilution); a mouse mAb against Neu N (Chemicon International, California, 1:100 dilution). The rabbit antiserum directed against the NCAM protein core was a site-directed antibody recognizing the seven NH-2-terminal residues of NCAM (1:1000 dilution) (See Rougon and Marshak, “Structural and immunological characterization of the amino-terminal domain of mammalian nueral cell adhesion molecules,” J. Biol. Chem., 261:3396-3401 (1986), which is incorporated by reference herein). O4 monoclonal antibody (hybridoma supernatant, 1:5 dilution) (described in Eisenbarth et al., 1979) was used to identify undifferentiated oligodendrocytes. Hoechst 33258 was used to counterstain cell nuclei in some cases. Fluorescence was examined with a fluorescence microscope (Axiophot; zeiss, Oberlochen, Germany). Controls treated with non-specific mouse IgM, or IgG preimmune sera or secondary antibody alone showed no staining. In double immunolabeling experiments, the use of only one primary antibody followed by the addition of both anti-mouse FITC and anti-rabbit TRITC-conjugated secondary antibodies resulted only in single labeling. Proliferating cells were identified with a monoclonal antibody against BrdU (Boehringer, 1:50 dilution) after 20-hour incorporation.

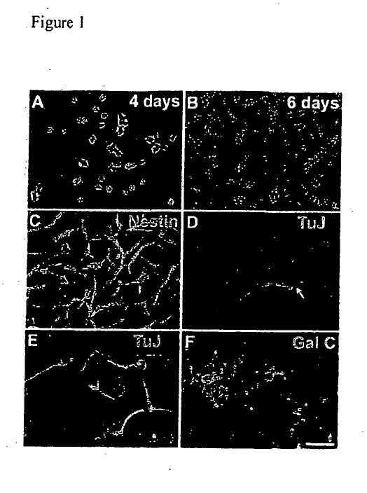

Four days after plating, the cells had an immature, round, or biopolar morphology as seen in FIG. 1A. Daily observations included that cells divided, formed loose colonies, and by day 6, formed a monolayer as seen in FIG. 1B. This monolayer may expose cells to FGF-2 more evenly and favor the formation of a homogenous population of undifferentiated progenitor cells. At this stage, the vast majority (98%) of the cells were stained with an anti-nestin antibody, as seen in FIG. 1C. Nestin is considered to be a marker for neural progenitor cells. Less than 3.2% of the cells expressed the neuronal marker Tuj. As seen in FIG. 1D, PSA-NCAM and BrdU incorporation showed that these cells did not divide. Very few to no cells displayed immunoreactivity for GFAP, or GalC, markers for astrocytes and oligodendrocytes, respectively. Their presence is probably due to contamination of the initial cell population after isolation and purification of progenitors. With the exception of a few differentiated cells, progenitor cells maintained in the presence of FGF-2 did not display antigenic markers for neuron- or glia-restricted precursor cells including PSA-NCAM, doublecortin, NeuN, NG2, or A2B5 (data not shown). In addition, nestin-positive cells were negative for endothelial markers such as von Willebrand factor and RECA-1 (data not shown). These results indicated that the cultures are immature cells that do not yet possess cell lineage-specific markers for neurons or glial cells.

When cultures were allowed to differentiate under conditions shown previously to stimulate both neuronal and glial differentiation (as described in Palmer et al., “The adult hippocampus contains primordial neural stem cells,” Mol. Cell. Neurosci., 8:389-404 (1997)), greater than 96% of the population displayed immunoreactivity for neuronal and astrocytic marker (Tuj+, 21%, GFAP+, 75%) as seen in FIG. 1E. The remaining population was immunoreactive for oligodendrocyte markers A2B5 or Gal C, as seen in FIG. 1F. These observations show that FGF-2 expanded cells are multi-potential neural progenitor cells that can give rise to neurons, astrocytes, and oligodendrocytes, the three major cell types in the central nervous system.

### Example 2

### Migration of FGF-2 Stimulated Neural Progenitor Cells are Modulated by a VEGFR-2 Ligand

Chemotaxis of neural progenitor cells was directly viewed and recorded in stable concentration gradients of VEGF (human recombinant, 165-amino acid homodimeric form, purchased from Peprotec Inc, Rochy Hill, N.J.) using the Dunn chemotaxis chamber (Weber Scientific international Ltd, Teddington, UK) (described in Zicha et al., “A new direct-viewing chemotaxis chamber,” J. Cell Sci., 99:769-775 (1991); Allen et al., “A role for Cdc42 in macrophage chemotaxis,” J. Cell. Biol, 141:1147-1157 (1998), both of which are incorporated by reference herein). Recombinant human VEGF-C_{ΔNΔC} (Dr. M. Skobe, Cancer Center, Mount Sinai Medical Center, New York) was used in some experiments. The Dunn chamber is made from a Helber bacteria counting chamber by grinding a circular well in the central platform to leave a 1 mm wide annular bridge between the inner and the outer well. Chemoattractants added to the outer well of the device will diffuse across the bridge to the inner blind well of the chamber and form a gradient. This apparatus allows one to determine the direction of migration in relation to the direction of the gradient.

Coverslips with cells were inverted onto the chamber and cell migration was recorded through the annular bridge between the concentric inner and outer wells, and a period of 2 hours was chosen to assess cell migration. In these studies, a systematic sampling was applied and all cells within the migration region of the chamber were recorded and analyzed. Data were recorded every 10 minutes using a ZEISS 10× objective via a HAMAMATSU CCD video camera using Openlab software.

In these chemotaxis experiments, the outer well of the Dunn chamber was filled with medium containing 200 ng/ml VEGF and 20 ng/ml FGF-2 and the concentric inner well with only medium and FGF-2. For chemokinesis experiments, VEGF (20 ng/ml) or FGF-2 (20 ng/ml) was added to both outer and inner wells of the Dunn chamber.

Directionality of cell movement was analyzed using scatter diagrams of cell displacement. The diagrams were oriented so that the position of the outer well of the chamber was vertically upwards (y direction). Each point represents the final positions of the cells at the end of the recording period where the starting point of migration is fixed at the intersection of the two axes.

To determine the efficiency of forward migration during the 2-hour recording period, each cell's forward migration index (FMI) was calculated as the ratio of forward progress (net distance the cell progressed in the direction of VEGF source) to the total path length (total distance the cell traveled through the field) (Foxman et al., 1999). FMI values were negative when cells moved away from the source of VEGF. The cell speed was calculated for each lapse interval recorded during the 2-hour period.

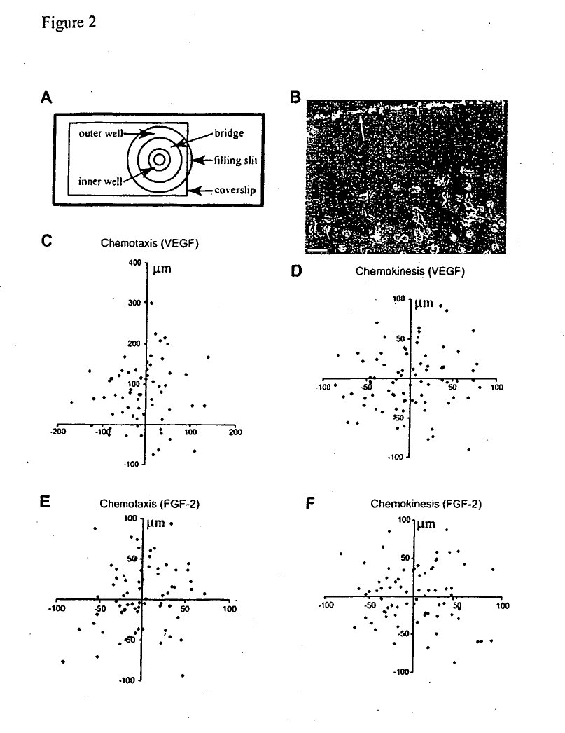

As shown in FIGS. 2A and 2B, chemoattractants added to the outer well of the Dunn chamber diffuse across the bridge to the inner well and form a linear steady gradient within ˜30 minutes of setting up the chamber. The gradient remains stable for ˜30 hours thereafter. Progenitor cells at day six maintained in the presence of FGF-2 and exposed to concentration gradients established with 200 ng/ml VEGF displayed strong positive chemotaxis as indicated in FIG. 2C. The scatter diagram of cell displacements in FIG. 2C demonstrates a strong directional bias of migration toward the source of VEGF. In contrast, when VEGF was added to both the inner and outer wells (chemokinesis conditions), cells remained motile by the population as a whole showed no clear preference for displacement as indicated in FIG. 2D. In these experiments, 20 ng/ml of FGF-2 was systematically included in the medium during the recording of neural progenitor chemotaxis or chemokinesis. However, FGF-2 had no chemotactic effect on these cells, irrespective of whether or not VEGF was present as indicated in FIGS. 2E and F. No difference was detected in the migratory behavior between cells exposed to an FGF-2 gradient, as indicated in FIG. 2E and cells exposed to a uniform concentration of FGF-2, as indicated in FIG. 2F.

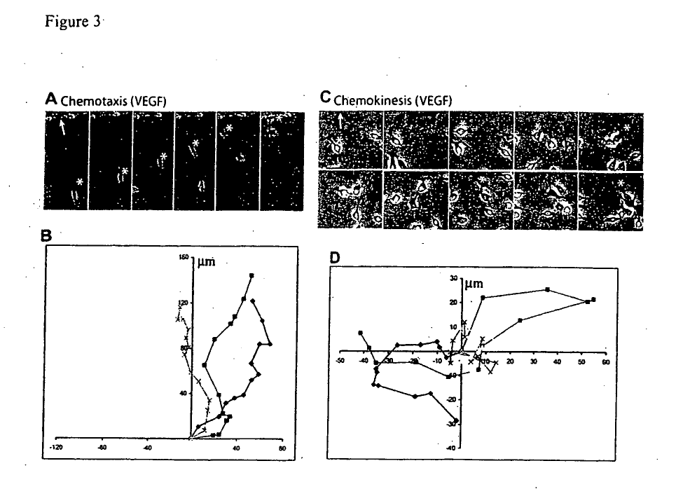

These observations were confirmed by the examination of individual cell tracks. As shown in FIG. 3, neural progenitor cells exposed to a VEGF gradient migrated efficiently toward the source of VEGF, as shown in FIGS. 3A and 3B, whereas those under conditions of chemokinesis, as shown in FIGS. 3C and D or exposed to an FGF-2 gradient made random turns during migration.

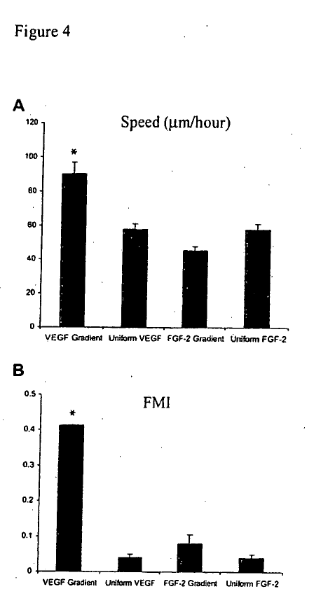

Referring to FIGS. 4A and B, quantitative analysis of the cells revealed that both migration speed (FIG. 4A), and the FMI (FIG. 4B) of cells exposed to VEGF in the presence of FGF-2 were significantly greater than those of cells exposed to an FGF-2 gradient or a uniform concentration gradient of VEGF or FGF0-2 (chemokinesis). The attractive effect of VEGF was similar on laminin-, poly-L-lysine-, or matrigel-coated coverslips. These data indicate that VEGF is an attractant for FGF-2 stimulated neural progenitor cells and this effect is matrix independent.

Similar results were obtained with VEGF-C_{ΔNΔC}.

### Example 3

### Neural Progenitor Cells Express VEGFRs

RNA Purification and RNase Protection Assay

Neural progenitor cells at 6 days of culture in FGF-2 or after starvation of FGF-2 for 12 hours were used for RNA preparation. Total cellular RNA was purified using Trizol reagent (Invitrogen). RNase protection assays were performed using cRNA probes for rat VEGFR1 and VEGFR2 as described in Pepper et al. (2000).

Immunoprecipitation and Western Blotting

Neural progenitor cells from the normal cultures in FGF-2 or from cultures starved of FGF-2 for 12 hours were lysed and VEGFR-2 protein was immunoprecipitated from cell lysates with a polyclonal antibody (sc-504; Santa Cruz Biochemicals, Santa Cruz, Calif.) recognizing amino aids 1158 to 1345 in the mouse VEGFR2 carboxy terminus. Western blot was performed with a polyclonal anti-VEGFR-2 antibody (sc-315; Santa Cruz Biochemicals) recognizing the mouse carboxy terminal amino acids 1348 to 1367.

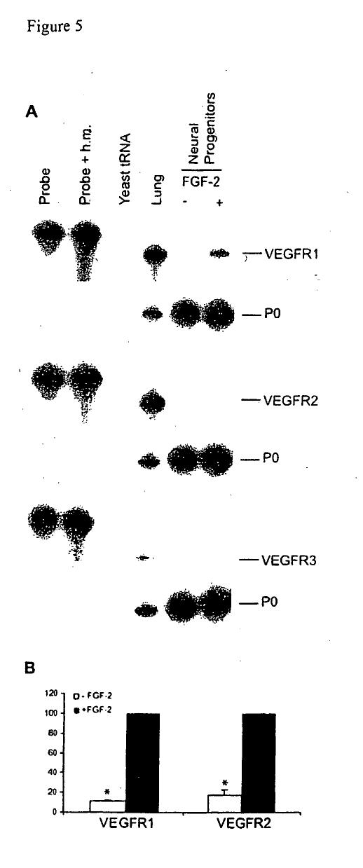

The FGF-2 stimulated neural progenitor cells expressed VEGFR-1 and VEGFR-2. mRNA for VEGFR-3 was not detected in these cultures as seen in FIG. 5A. After 12 hours of starvation of FGF-2, there was a marked, fivefold decrease in the level of VEGFR-1 and VEGFR-2 transcripts as shown in FIGS. 5A and B. These results demonstrate that FGF-2 stimulated neural progenitor cells express mRNA for both VEGFR-1 and VEGFR-2, but not VEGFR-3 and that FGF-2 is required for this expression

It is unlikely that down-regulation of VEGF receptor expression and the lack of chemotactic responses are due to death or suffering of cells in the absence of FGF-2, which is demonstrated by the following: 1) after removal of FGF-2 for 12 hours, cells maintained in neurobasal medium supplemented with B27 displayed no difference in morphology compared to control cultures; 2) Hoechst 33258 staining of cell nuclei did not reveal any difference between cultures kept in the presence or absence of FGF-2; 3) video analysis revealed that cells in the absence the FGF-2 exhibited random migration with the same migration speed as control cells in the presence of FGF-2; 4) FGF-2 starvation did not change the expression of acidic ribosomal phosphoprotein (P0). In vitro, FGF-2 is known to stimulate mitotic activity in progenitors cells and to maintain these cells in an undifferentiated state (Palmer et al., 1997; Tropepe et al., 1999). Since withdrawal of FGF-2 from cultures is a standard procedure used to induce the differentiation of FGF-2-stimulated progenitors (Palmer et al., 1997; Tropepe et al., 1999), the more differentiated progenitors may loose VEGFR expression as well as the capacity to respond to VEGF. However, the effect of FGF-2 withdrawal was reversible upon the re-application of FGF-2 to the medium after 8 hours. VEGF receptor expression may also be induced by FGF-2 in differentiated neurons.

### Example 4

### VEGFR-2 Ligand-Induced Chemotaxis is Mediated Through VEGFR-2

MF1, a VEGFR1 blocking antibody and DC101, a VEGFR2 blocking antibody (ImClone Systems Incorporated, New York) were both added at 20 μg/ml to the neural progenitor cells after the steps of Example 2 and were used to block the function of the corresponding VEGF receptor. A polysialic acid blocking antibody was used as a control.

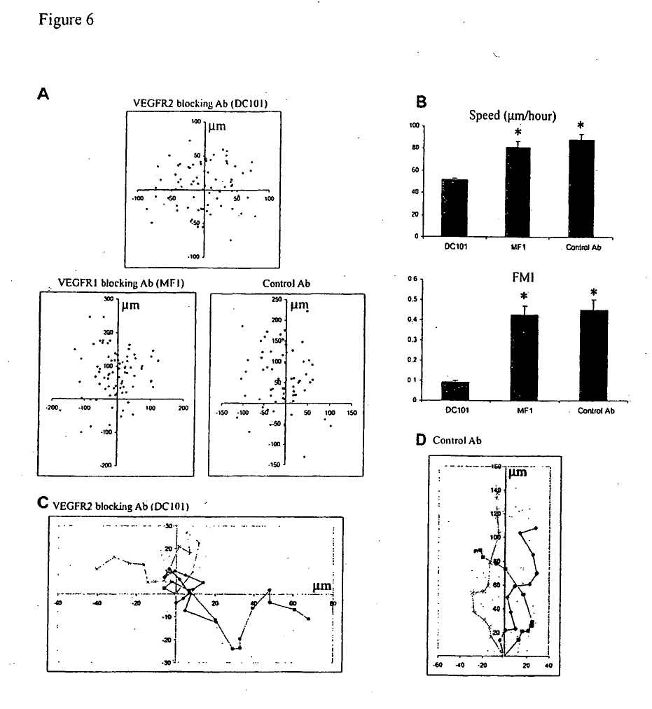

As indicated in FIGS. 6A and C, the chemotactic response of cells to VEGF was completely abrogated by DC101. In contrast, the MF1 did not affect chemotaxis as indicated in FIG. 6A. These observations were confirmed by measurements of speed and FMI as indicated in FIG. 6B. In the absence of a VEGF gradient, the addition of anti-VEGFR-2 had no significant effect on neural progenitor cell migration. These experiments demonstrate that VEGF stimulates chemotaxis of progenitor cells through VEGFR-2.

This conclusion received further support from experiments in which concentration of VEGF-C_{ΔNΔC} was used to induce chemotaxis. It was observed that VEGF-C_{ΔNΔC} could efficiently induce chemotaxis of progenitor cells and that this effect was prevented by the VEGFR2 blocking antibody (data not shown). Furthermore, since VEGF-C_{ΔNΔC} exerts its function through VEGFR-2 and VEGFR-3, and since VEGFR3 is not expressed by FGF-2-stimulated neural progenitor cells, these results strengthen the conclusion that signaling through VEGFR-2 mediates chemoattraction of progenitor cells by VEGF.

### Example 5

### FGF-2 is required for a VEGF-2 Ligand to Stimulate Chemotaxis of Neural Progenitor Cells

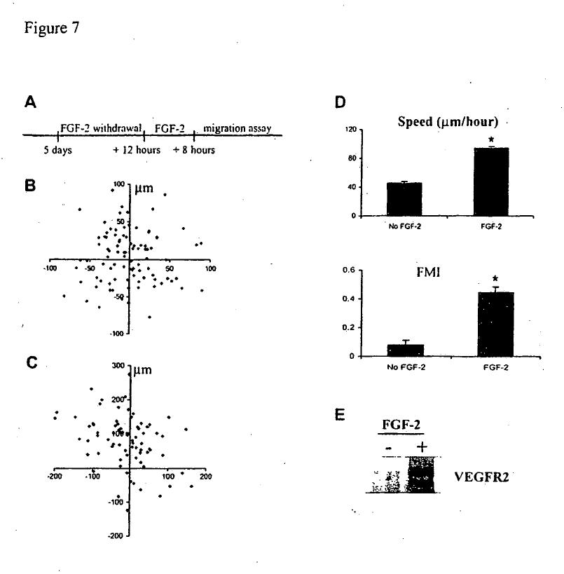

The migratory response of progenitors to VEGF in the absence of FGF-2 was examined. Cells at 5 days of culture were starved of FGF-2 for 12 hours and then exposed to a VEGF gradient (See Example 3). As shown in FIG. 7B, starved cells failed to undergo chemotaxis in response to VEGF. Cells migrated randomly in a manner similar to when they were exposed to a uniform concentration of VEGF. In agreement with these results, and confirming the data of the RNase protection assay, shown in FIG. 5 and described in Example 3, Western blot analysis revealed little to no expression of VEGFR-2 protein in the absence of FGF-2, while substantial expression was detected in the presence of FGF-2, as shown in FIG. 7E.

To determine whether the effect of FGF-2 withdrawal is reversible and whether cells could chemotactically respond to VEGF upon re-addition of FGF-2 to the cultures, FGF-2 was included in the medium after a 12-hour starvation period and the cells were further cultured for 8 hours. Diagrams of displacements of motile cells shown in FIG. 7C and a quantitative analysis of forward migration index and speed, shown in FIG. 7D demonstrated that the loss of chemotaxis was rescued after an 8-hour re-incubation with FGF-2. Taken together, these data demonstrate that FGF-2 is necessary for the expression of VEGFR2 and for an adequate migratory response of progenitors to concentration gradients of VEGF.

### Example 6

### VEGF-2 Ligand Affects Migration of Neural Progenitor Cells from the Subventricular Zone

The frontal lobes of the brains of one-day-old Sprague-Dawley rat pups (Size, Zurich, Switzerland) were isolated and cut into 300 μm thick coronal sections with a McIllwain tissue chopper. From these slices the anterior part of the subventricular zone (SVZ) was microdissected. The SVZ explants were embedded in a collagen matrix and cultured for 7 days in chemically-defined serum-free medium (50% Dulbecco's modified Eagle's medium [Gibco, Berlin, Germay], 50% F12, HEPES, Tris-HCl, and complemented with transferrin human 20 μg/ml, putrescine 100 μM, sodium selenite 30 nM, triiodothyronin 1 nM, docosahexaenoic acid 0.5 μg/ml, arachidonic acid 1 μg/ml, insulin 60 U/l) under 5% CO₂. The medium was changed every 3^{rd} day. For co-culture experiments, SVZ explants were cultured in the presence of murine C₂C₁₂ myoblasts that had been engineered to secret VEGF (Rinsch et al., 2001). C₂C₁₂ cells were suspended in a drop of collagen matrix which was placed at a distance of approximately 1,000 μm from the SVZ explant. As a control, mock-transfected cells of the same origin were placed into the collagen matrix in a similar manner and at the same distance, but on the opposite side of the explant.

Cell migration was assessed at the end of the 7^{th} day in culture. Three categories were established: 1, no migration: no or only a few cells emigrated from the explants; 2, symmetrical migration: numerous cells had left the explants, the distance of the migrating front of the cells exceeded 50 μm, no directionality of migration; 3, asymmetrical or directional migration: when the distance of the migrating front were at least twice that on the other side and exceeded 50 μm.

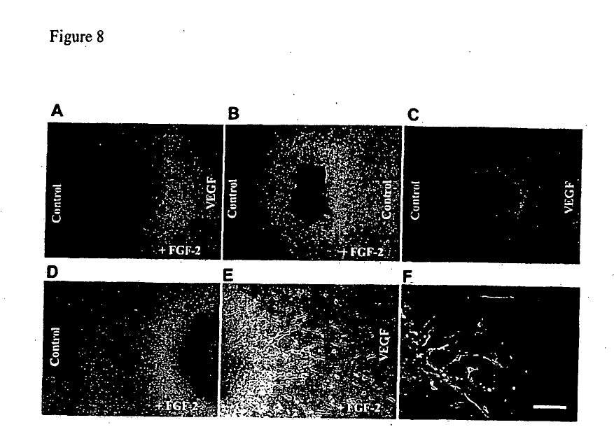

As shown in FIGS. 8B and 8D, when explants were co-cultured with aggregates of mock-transfected cells in the presence of FGF-2 (20 ng/ml), migrating cells were symmetrically distributed around the explants (10/10 explants). As shown in FIGS. 8A and 8E, when SVZ explants were co-cultured, in the presence of FGF-2, with VEGF-expressing cells placed on one side and with mock-transfected cells on the other, cell migration was highly asymmetric (10/20 explants with cells migrating predominantly towards VEGF-secreting C₂C₁₂ cells, and, 10/20 explants with a symmetric migratory pattern). As shown in FIG. 8C. In contrast, when explants were co-cultured with control or VEGF-expressing cells in the absence of FGF-2, no significant cell migration from SVZ explants was observed (10/10 explants). Similar results were obtained after application of VEGF in the absence of FGF-2 (4/4 explants). The application of VEGF and FGF-2 together or FGF-2 alone resulted in symmetric migration (12/12). To determine whether cells migrating in response to VEGF are immature progenitors, immunocytochemical staining with an anti-nestin Ab was carried out. Migrating cells stained positively for nestin, as seen in FIG. 8F and were negative for PSA-NCAM (a marker for immature neurons, not shown), confirming that they were indeed immature progenitor cells. Together, these results indicate that immature progenitor cells migrate in response to VEGF gradients, and that FGF-2 is required for this effect.

The foregoing description and examples have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

We claim:

1. A method for modulating the migration of neural progenitor cells comprising exposing the cells to FGF-2 and a VEGFR-2 ligand.

2. The method of claim 1, wherein the cells are exposed to the FGF-2 prior to exposure to the VEGFR-2 ligand.

3. The method of claim 1, wherein the VEGFR-2 ligand is selected from the group consisting of VEGF, VEGF-E, and VEGF-C/D_{ΔNΔC}.

4. A method for treating a mammal having a disorder involving loss or injury of neural cells comprising exposing the mammal to a VEGFR-2 ligand and FGF-2 to stimulate migration of neural progenitor cells to the site of neural cell loss or injury.

5. The method of claim 4, wherein exposing the mammal to a VEGFR-2 ligand comprises administering a VEGFR-2 ligand to the mammal.

6. The method of claim 4, wherein the VEGFR-2 ligand is selected from the group consisting of VEGF, VEGF-E, and VEGF-C/D_{ΔNΔC}.

7. The method of claim 4, wherein the FGF-2, the VEGFR-2 ligand, or both are administered to the site of neural cell loss or injury.

8. The method of claim 4, wherein the neural progenitor cells are transplanted into the mammal.

9. The method of claim 8, wherein the cells express VEGFR-1 and VEGFR-2.

10. The method of claim 8, wherein the cells do not express PSA-NCAM, doublecortin, NeuN, NG2, A2B5, von Willebrand factor, RECA-1, or any combination thereof.

11. The method of claim 4, wherein the disorder involving loss or injury of neural cells is brain injury.

12. The method of claim 11, wherein the brain injury is produced by head trauma, stroke, anoxia, or ischemia.

13. The method of claim 4, wherein the FGF-2 is associated with a biocompatible matrix.

14. The method of claim 4, wherein the VEGFR-2 ligand is associated with a biocompatible matrix.

15. A method for treating a mammal having a neural tissue site with a deficient neuronal population comprising exposing the mammal to a VEGFR-2 ligand in the presence of FGF-2 to stimulate migration of neural progenitor cells to the neural tissue site.

16. The method of claim 15, wherein exposing the mammal to a VEGFR-2 ligand comprises administering a VEGFR-2 ligand to the mammal.

17. The method of claim 15, wherein the VEGFR-2 ligand is selected from the group consisting of VEGF, VEGF-E, and VEGF-C/D_{ΔNΔC}.

18. A method for modulating the migration of neural progenitor cells comprising exposing the cells to a VEGFR-2 ligand and a compound capable of increasing the expression of VEGFR-2 on the cells.

19. The method of claim 18, wherein the compound is FGF-2.

20. A composition comprising a biocompatible matrix comprising FGF-2.

21. The composition of claim 20, wherein the biocompatible matrix further comprises a VEGFR-2 ligand.

22. The composition of claim 20, further comprising neural progenitor cells.

23. The composition of claim 22, wherein the cells express VEGFR-1 and VEGFR-2.

24. The composition of claim 22, wherein the cells do not express PSA-NCAM, doublecortin, NeuN, NG2, A2B5, von Willebrand factor, RECA-1, or any combination thereof.

25. A pharmaceutical composition comprising a VEGFR-2 ligand, FGF-2 and a carrier.

26. The composition of claim 25, further comprising neural progenitor cells.
